(19) United States
(12) Patent Application Publication
(10) Pub. No.: US 2012/0135460 A1
(43) Pub. Date: May 31, 2012

(54) RECOMBINANT PORCINE CHYMOTRYPSIN

(75) Inventors: Raymond-David Pridmore, St-Sulpice (CH); Fabrizio Arigoni, Tokyo (JP); Francoise Maynard, Speigel Bei Bern (CH); Isabelle Bureau-Franz, Epalinges (CH)

(73) Assignee: NESTEC S.A., Vevey (CH)

(21) Appl. No.: 13/140,726

(22) PCT Filed: Dec. 11, 2009
(86) PCT No.: PCT/EP2009/066904

§ 371 Date: Sep. 1, 2011

(30) Foreign Application Priority Data
Jan. 6, 2009 (EP) 09150114.8

(51) Int. Cl. C12P 21/06; C12N 15/57; C12N 1/15; C12N 1/21; C12N 1/19; C12N 5/10; C12N 9/76; C12N 15/63

(52) U.S. Cl. 435/68.1; 435/213; 536/23.2; 435/320.1; 435/252.3; 435/254.2; 435/419; 435/348; 435/325; 435/254.11

(57) ABSTRACT

The present invention generally relates to the field of proteinases and more specifically to chymotrypsin. In particular, the present invention relates to recombinant porcine chymotrypsin and its use in food applications.

The present invention generally relates to the field of proteinases and more specifically to chymotrypsin. In particular, the present invention relates to recombinant porcine chymotrypsin and its use in food applications.

Food products typically contain a protein source.

While generally these protein sources are used in a form that nature provides, there are cases where it might be preferred to add a protein sources in a modified form to a food composition.

For example, in subjects with compromised functioning of the gastro-intestinal tract it

preferred, if the subject ingests a diet with short peptide chains to facilitate absorption and food tolerance. For example, nutritional compositions with short peptide chains, such as Peptamen®, has been shown to reduce the incidence of diarrhoea to 0% compared to 40% in ICU patients receiving an intact protein formula (Meredith et al J Trauma 1990; 30:825-829).

Such a nutritional composition is in particular appropriate for the metabolically stressed children, those with compromised gastro-intestinal function and those with challenging feeding issues.

Peptamen® contains as a protein source peptides from hydrolysed whey protein, which provide an easily absorbed and well utilised source of nitrogen. These whey derived peptides are even better absorbed than free amino acids.

Hydrolysed proteins may also be used by subjects that suffer from allergic disorders. Food allergies, of which the first to occur in life is cows' milk allergy, are caused, in most cases, by a reaction to the proteins in the food. In the early years of life the immune system is still developing and may fail to develop tolerance to dietary antigens (this may also be described as insufficient induction of oral tolerance). The result is that the baby or child or young animal mounts an exaggerated immune response to the dietary protein and develops an allergic response to it. Food allergies may affect not only humans but also other mammals such as dogs and cats. Usually, food hypersensitivity appears just after a susceptible baby, child or young animal first encounters a new food containing potential allergens. Apart from its mother's milk, the first dietary proteins generally encountered by human babies at least are cows' milk proteins and, as noted above, cows' milk allergy is the most common food allergy in human babies. It is generally accepted that babies with established cows' milk allergy have an increased risk of developing atopic diseases and allergies to other dietary proteins such as egg and cereal proteins but even those babies who have successfully developed oral tolerance to cows' milk proteins may subsequently develop allergies to other dietary proteins such as egg and cereal proteins when these are introduced into the diet at weaning. These allergies may manifest themselves clinically as atopic diseases such as atopic dermatitis, eczema and asthma. From a dietary point of view there are two ways to treat an established allergy—either foods containing the allergen must be avoided altogether, or the foods must be treated to decrease their allergenic potential, for example by extensive hydrolysis. Infant formulas containing extensively hydrolysed cows' milk proteins (peptides consisting of not more than five amino acids) are manufactured for this latter purpose. Similarly it has already been proposed, in U.S. Pat. No. 6,403,142 for example, to prepare pet foods with reduced allergenicity for companion animals where it is suspected that the animal has developed a food allergy.

Partially hydrolysed proteins may also be used to induce oral tolerance. Products have been devised which help to reduce the risk of developing the allergy in the first place, particularly for children thought to be at risk of the same (that is, children having at least one close family member who suffers from an allergy). One example of such products is the infant formulas based on partially hydrolysed whey proteins sold under the trade marks NAN HA1 and NAN HA2. These products have been demonstrated to actively induce oral tolerance to cows' milk proteins. Fritsche et al. (J. Allergy Clin. Immunol, Vol 100, No. 2, pages 266-273, 1997) have shown using animal models that enzymatic hydrolysates of cow's milk proteins with a degree of hydrolysis of 18% were able to induce oral tolerance to intact cow's milk proteins whereas hydrolysates with a degree of hydrolysis of 28% were not. Results of these experiments showed that preventive feeding of rats with such a moderately hydrolysed cow's milk formula, whose allergenicity had been reduced over 100 times as compared to a standard formula, suppressed specific IgE and mediator release from intestinal mast cells, both parameters of an immediate type allergic reaction. This work demonstrated that for cows' milk proteins it is possible to define a degree of enzymatic hydrolysis whereby the capacity of the peptides to induce oral tolerance is maintained whilst their allergenicity is substantially reduced.

Typically, protein sources are modified in the food industry today by the use of enzymes that are obtained from natural sources.

For example, milk protein or milk derived proteins are often hydrolysed today using animal chymotrypsin.

Chymotrypsin is often obtained from porcine sources, for example from the porcine pancreas. However using enzymes purified from animal parts involves the sacrifying animals. Further, chymotrypsin needs to be purified, which is both, labour and time consuming as well as expensive. The resulting chymotrypsin preparation after purification might still contain residual impurities, for example other proteinases, such as trypsin.

It was the object of the present invention to overcome these disadvantages of the prior art and to provide the art with chymotrypsin that corresponds with respect to its activity to porcine chymotrypsin but that does not have to be isolated from porcine sources.

The present inventors were surprised to see that they could achieve this object by a chymotrypsin in accordance with claim 1, a DNA in accordance with claim 4, and a use in accordance with claim 10.

The present inventors have discovered that porcine chymotrypsin can alternatively be produced by using biotechnological methods.

Consequently, the present invention relates to a recombinant porcine chymotrypsin.

For the purpose of the present invention the term “chymotrypsin” is meant to include both, the active chymotrypsin as well as the inactive precursor chymotrypsinogen.

The gene for porcine chymotrypsin may be, for example amplified by PCR from porcine DNA or, alternatively, may be synthesized and provided in the form of a synthetic gene.

Synthetic genes are commercially available. One preferred embodiment of the present invention, hence, relates to recombinant porcine chymotrypsin obtained from a synthetic gene. Obtaining recombinant porcine chymotrypsin from a synthetic gene has for example the advantage that no porcine material needs to be used for the production of recombinant porcine chymotrypsin. This would render the resulting enzyme acceptable for parts of the population that cannot tolerate porcine material in their food products.

Also, the present invention includes recombinant porcine chymotrypsin synthesized in vitro from a corresponding amino acid sequence.

The porcine chymotrypsin may be chymotrypsin B or chymotrypsin C, for example.

Obviously, alterations to the protein and/or DNA sequence of porcine chymotrypsin or the porcine chymotrypsin gene may be made, for example to optimize enzyme activity, to allow an expression in higher yields, and/or to optimize the gene for expression in a particular organism, for example by optimizing codon usage. One embodiment of the present invention relates hence to recombinant porcine chymotrypsin, wherein a part of the chymotrypsin gene and/or protein is replaced, deleted, or added, while the protein still has at least 80%, preferably at least 90%, even more preferred at least 98% of the activity of porcine chymotrypsin.

In terms of amino acid similarity, the recombinant chymotrypsin of the present invention comprises preferably an amino acid sequence with greater than 90% similarity, preferably greater than 95% similarity, more preferably more than 99% similarity to the natural amino acids sequence of porcine chymotrypsin. In the context of the present invention, amino acids sequence with more than 90% similarity means at least 90% identical or conservatively replaced amino acid residues in a like position when aligned optimally allowing for up to 4 gaps with the proviso that in respect of each gap a total of not more than 10 amino acid residues is affected.

The amino acid substitutions are preferably conservative substitutions. Examples of the conservative substitutions of naturally occurring amino acids include aliphatic amino acids (Gly, Ala, and Pro), hydrophobic amino acids (lie, Leu, and Val), aromatic amino acids (Phe, Tyr, and Trp), acidic amino acids (Asp, and Glu), basic amino acids (His, Lys, Arg, Gln, and Asn), and sulfur-containing amino acids (Cys, and Met). The deletions of amino acids are located preferably in a region which is not involved directly in the active site of porcine chymotrypsin.

In terms of nucleic acid sequence similarity, the recombinant chymotrypsin gene of the present invention comprises preferably a DNA sequence with greater than 80% identity, preferably, greater than 90% identity, more preferably, more than 95% identity to the natural DNA sequence of the porcine chymotrypsin gene.

As used herein, the term “nucleic acid sequence” is intended to include DNA, mRNA, complementary DNA (cDNA) sequence and equivalent nucleic acid sequences thereof. As used herein, the term “equivalent nucleic acid sequence” is intended to include sequences with allelic variation or degenerate codon sequences. As used herein, the term “degenerate codon sequence” refers to a nucleic acid sequence, which is different from the naturally occurring sequence, but encodes a protein having the same sequence as porcine chymotrypsin. As a result of the degeneracy of the genetic code, nucleotide sequences encoding porcine chymotrypsin can be prepared diversely.

One embodiment of the present invention relates to DNA comprising at least one recombinant and/or synthetic gene coding for porcine chymotrypsin.

The present invention also provides a vector, preferably an expression vector comprising the recombinant gene of porcine chymotrypsin. Hence, the present invention also relates to a DNA comprising at least one recombinant and/or synthetic gene coding for porcine chymotrypsin, in the form of an expression vector.

The DNA comprising at least one recombinant and/or synthetic gene coding for porcine chymotrypsin, for example the vector or the expression vector, may contain at least one recombinant and/or synthetic gene coding for porcine chymotrypsin wherein a part of the chymotrypsin gene is replaced, deleted, or added, while the expressed protein still has at least 80% of the activity of porcine chymotrypsin.

As used herein, the term “vector” means a nucleic acid molecule that can carry another nucleic acid bound thereto. The term “vector” comprises for example any vehicle used to transfer foreign genetic material into another cell. As used herein, the term “expression vector” is intended to include a plasmid, cosmid or phage, which can be used to synthesize a protein encoded by a recombinant gene carried by said vector. Expression vectors are consequently typically used for the expression of the transgene in a target cell, and may comprise a promoter sequence that drives the expression of the transgene. A preferred vector is a vector that can self-replicate.

The present invention provides a method for producing recombinant porcine chymotrypsin. Methods to produce a protein from a synthetic or recombinant gene are known in the art. For example the protein of the present invention may be expressed in a cell free expression system. Alternatively, the protein may also be produced by transferring the gene encoding recombinant porcine chymotrypsin into a host cell, so that the host cell expresses the porcine chymotrypsin, optionally after induction. The gene may be functionally incorporated into the genome of the host cell. Alternatively, the gene may also be inserted into the host cell in the framework of an expression vector.

One embodiment of the present invention relates to a cell containing a DNA comprising at least one recombinant and/or synthetic gene coding for porcine chymotrypsin capable of expressing the protein coded for by the DNA. Hence, the present invention provides a host cell transformed with said recombinant vector.

As used herein, the term “transformation” means that foreign DNA or RNA is absorbed into cells to change the genotype of the cells. Host cells suitable for transformation include for example prokaryotic, yeast, fungal, plant and animal cells, but are not limited thereto. Most preferably, E. coli cells are used. Methods for culturing E. coli are well known in the art.

For example in one embodiment of the present invention, the cell is a micro-organism, for example a bacterial cell, a yeast cell, a plant cell, a fungi cell, an insect cell or a mammalian cell.

In a particular preferred embodiment of the present invention, the micro-organism is a food grade micro-organism. This has the advantage, that recombinant porcine chymotrypsin can be used on food products, for example as a fraction of a bacterial culture.

“Food-grade” means a material that is approved for human or animal consumption.

The recombinant porcine chymotrypsin of the present invention may be used for every application chymotrypsin can be used for.

In particular in the food industry the recombinant porcine chymotrypsin of the present invention and/or a cell culture containing a cell expressing recombinant porcine chymotrypsin or a fraction thereof comprising chymotrypsin activity may be used to at least partially digest a protein containing foodstuff or fractions thereof.

Protein containing foodstuffs include a food product, an animal food product or a pharmaceutical composition. For example, the product may be a nutritional composition, a nutraceutical, a drink, a food additive or a medicament.

A food additive or a medicament may be in the form of tablets, capsules, pastilles or a liquid for example. They may further contain protective hydrocolloids (such as gums, proteins, modified starches), binders, film forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. They may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, they may contain an organic or inorganic carrier material suitable for oral or enteral administration as well as vitamins, minerals trace elements and other micronutrients in accordance with the recommendations of Government bodies such as the USRDA.

For example milk derived proteins may be digested by the recombinant porcine chymotrypsin of the present invention. Milk includes cows' milk, human milk or soy milk, for example. Preferred milk proteins or milk protein fractions in accordance with the present invention comprise whey proteins, α-lactalbumin, β-lactoglobulin, bovine serum albumin, casein acid, caseinates, or α, β, κ-casein, for example.

The protein fractions that are at least partially digested by the recombinant porcine chymotrypsin of the present invention may be used to improve the bodies' ability to absorb the protein fraction after ingestion.

For example, the recombinant porcine chymotrypsin of the present invention may be used to at least partially digest protein fractions obtainable from milk to improve the bodies' ability to absorb the protein fraction after ingestion.

Those skilled in the art will understand that they can freely combine all features of the present invention described herein, without departing from the scope of the invention as disclosed. In particular, features described for the uses of the present invention may be applied to the foodstuff of the present invention and vice versa.

Further advantages and features of the present invention are apparent from the following sequence listing, examples and figures.

The sequence listing shows

SEQ-ID NO 1: Porcine cationic trypsinogen protein
SEQ-ID NO 2: Anionic trypsinogen protein
SEQ-ID NO 3: Chymotrypsinogen B protein
SEQ-ID NO 4: Chymotrypsinogen C protein
SEQ-ID NO 5: Intein-cationic trypsinogen fusion protein sequence
SEQ-ID NO 6: Intein-anionic trypsinogen fusion protein sequence
SEQ-ID NO 7: Intein-Chymotrypsinogen B fusion protein sequence
SEQ-ID NO 8: Intein-Chymotrypsinogen C fusion protein sequence
SEQ-ID NO 9: Synthetic cationic trypsinogen gene sequence
SEQ-ID NO 10: Synthetic anionic trypsinogen gene sequence
SEQ-ID NO 11: Synthetic Chymotrypsinogen B gene sequence
SEQ-ID NO 12: Synthetic Chymotrypsinogen C gene sequence

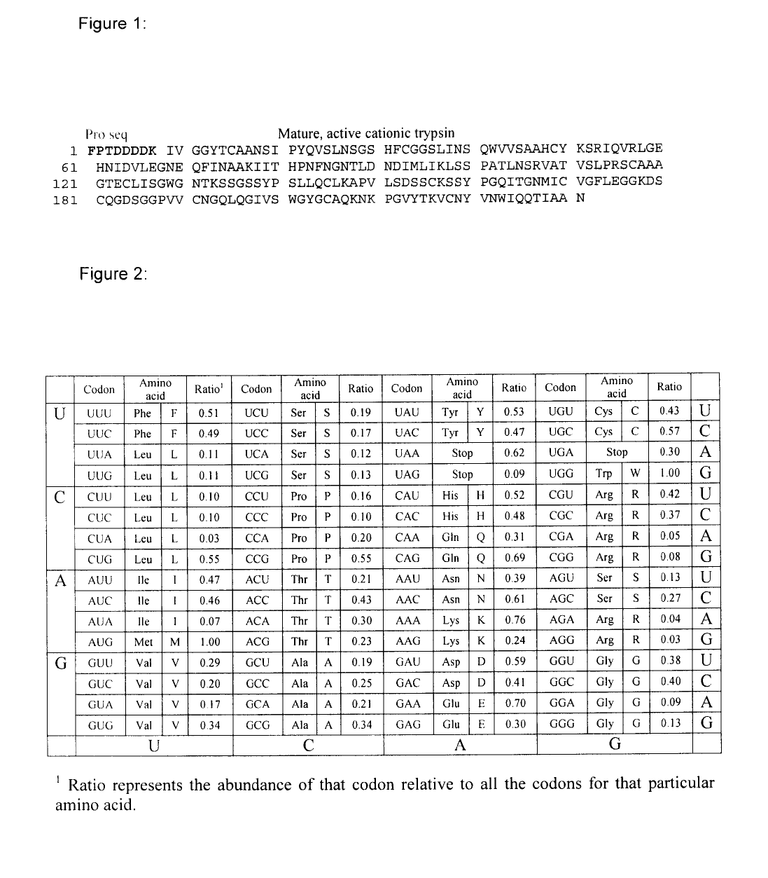

FIG. 1 shows the porcine cationic trypsinogen sequence from P00761 (231 aa).

FIG. 2 shows the codon usage table for Escherichia coli as modified from Maloy, S., V. Stewart, and R. Taylor. 1996. Genetic analysis of pathogenic bacteria. Cold Spring Harbor Laboratory Press, NY.

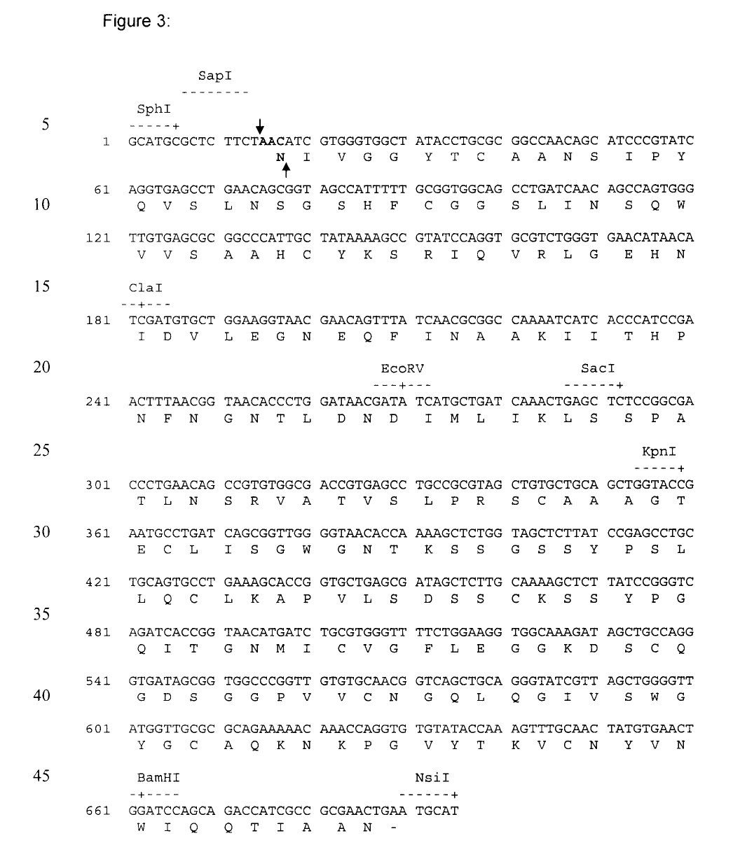

FIG. 3 shows the synthetic cationic trypsinogen gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

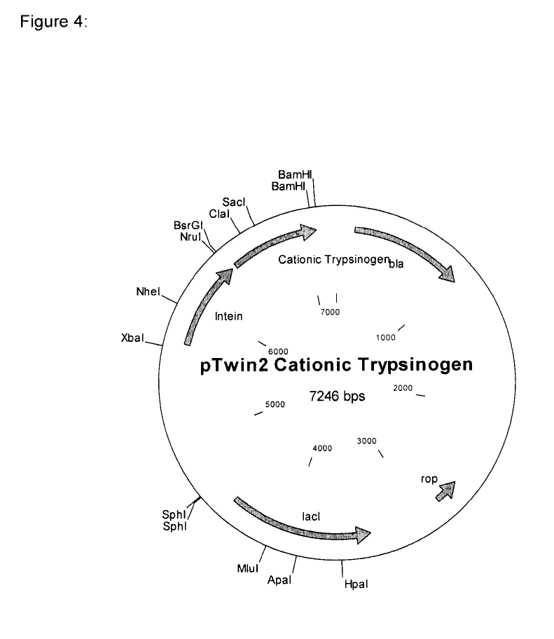

FIG. 4 shows a plasmid map of pTwin2-Cationic-trypsinogen for the expression of the fused intein-trypsin protein.

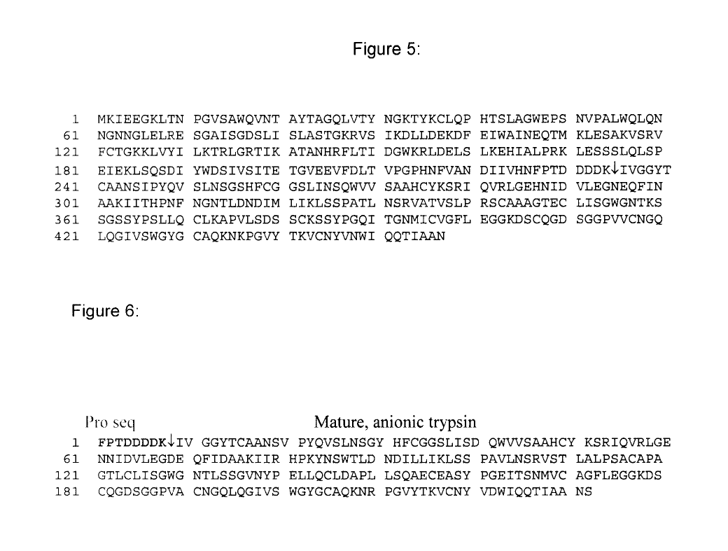

FIG. 5 shows an intein-cationic trypsinogen fusion protein sequence. The intein sequences are shown in red and the porcine trypsinogen in black.

FIG. 6 shows the porcine anionic trypsinogen sequence (232 aa).

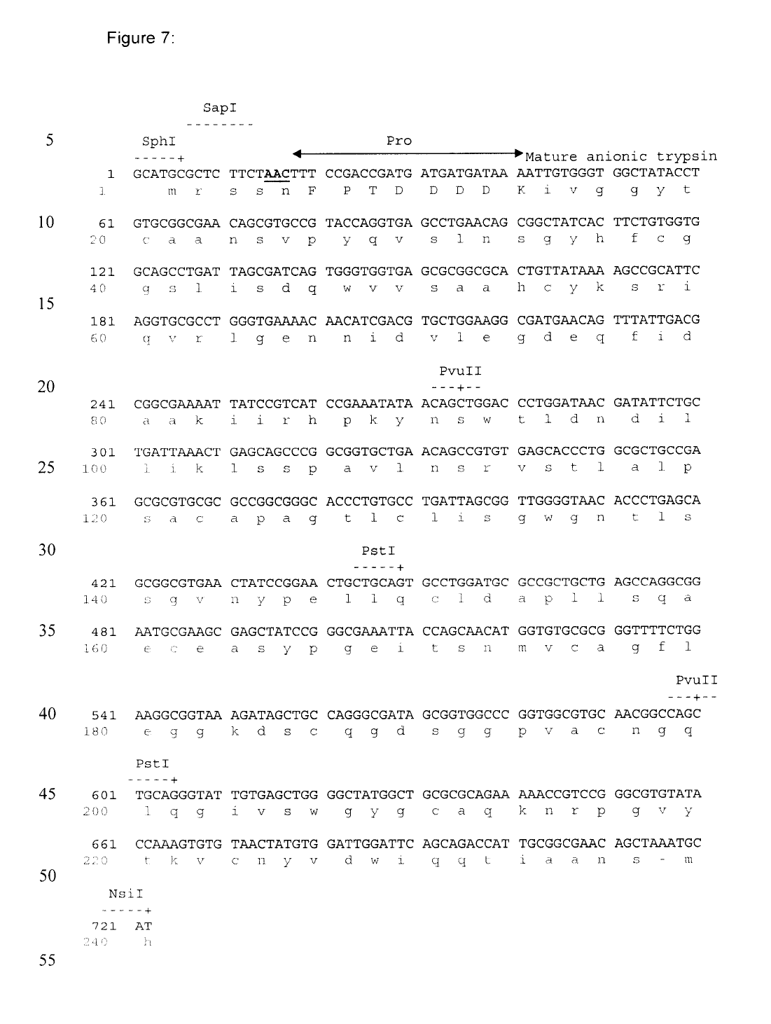

FIG. 7 shows the synthetic anionic trypsinogen gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

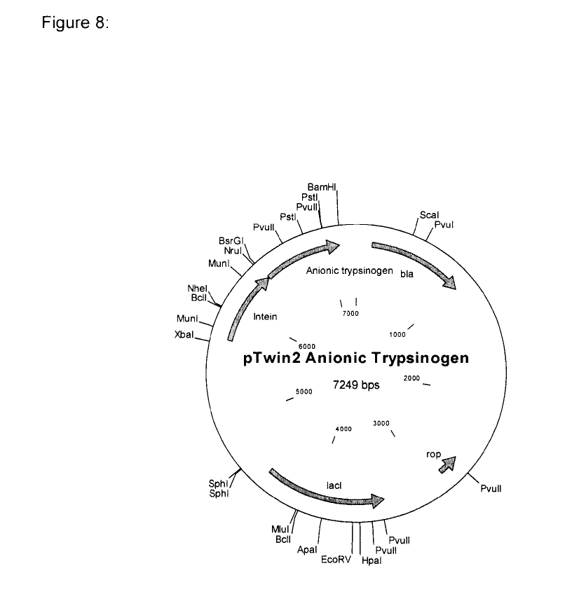

FIG. 8 shows a plasmid map of pTwin2-anionic trypsinogen for the expression of the fused intein-trypsin protein.

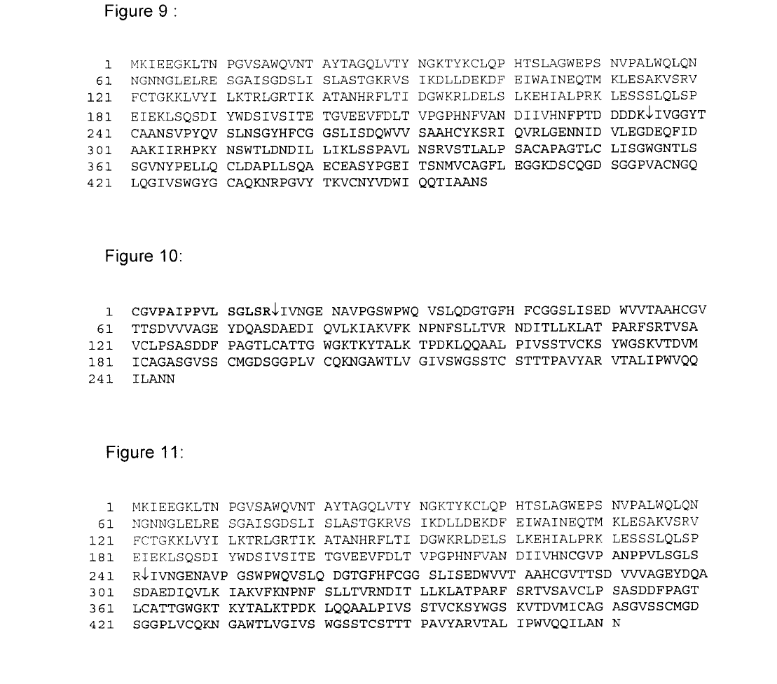

FIG. 9 shows the Intein-anionic trypsinogen fusion protein sequence. The intein sequences are shown in red and the porcine cationic trypsinogen in black.

FIG. 10 shows the chymotrypsinogen B sequence.

FIG. 11 shows an intein-chymotrypsinogen B fusion protein sequence. The intein sequences are shown in red and the porcine chymotrypsinogen B in black.

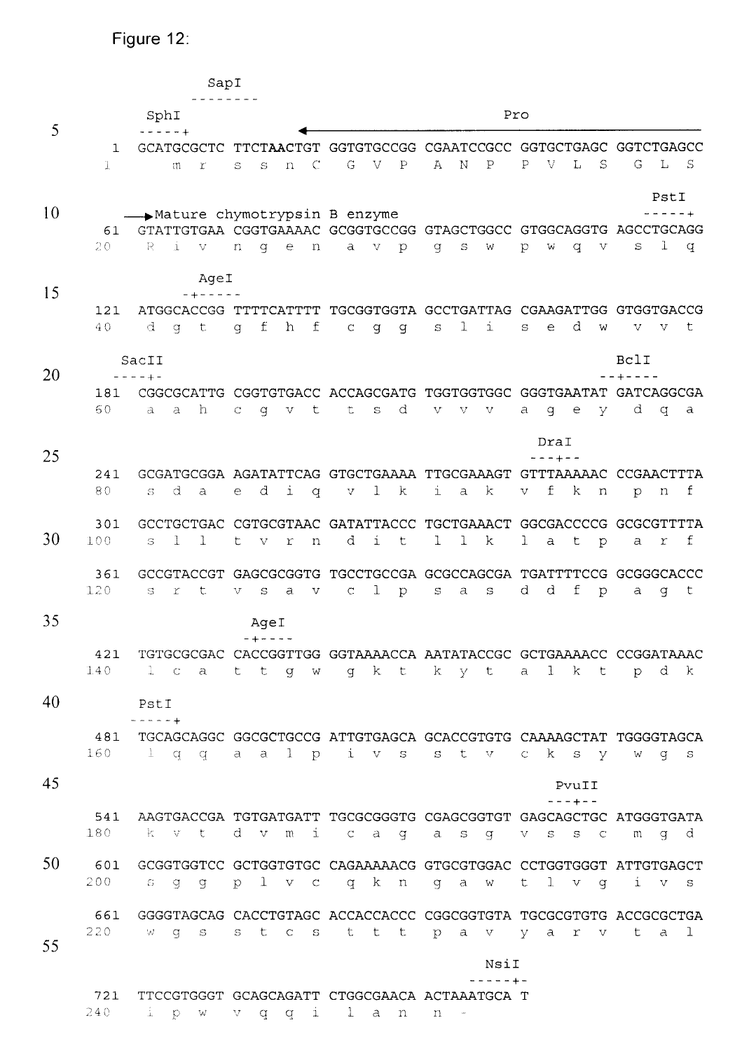

FIG. 12 shows the synthetic chymotrypsinogen B gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

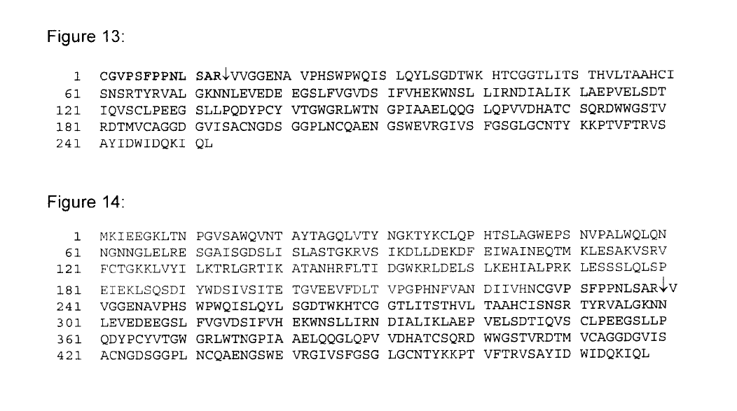

FIG. 13 shows the chymotrypsinogen C sequence.

FIG. 14 shows an intein-chymotrypsinogen C fusion protein sequence. The intein sequences are shown in red and the porcine chymotrypsinogen C in black.

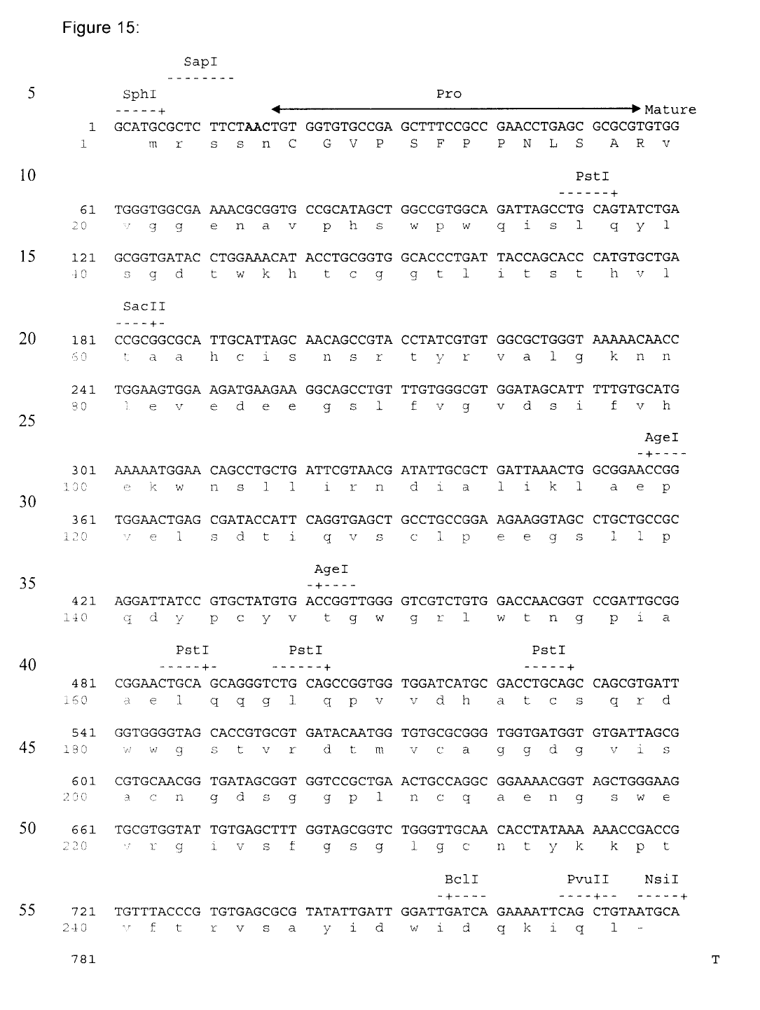

FIG. 15 shows the synthetic chymotrypsinogen C gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

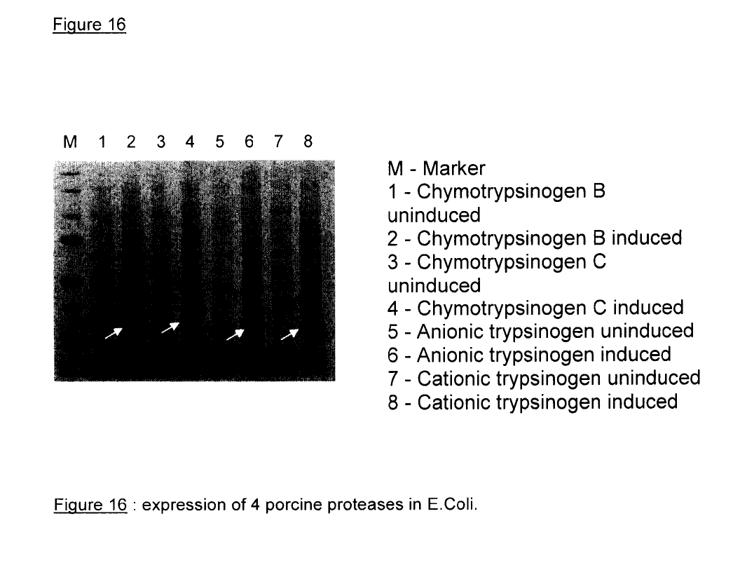

FIG. 16 shows the expression of the 4 porcine proteases in E. coliu: Lane 1 shows the insoluble cell wall associated proteins for the chymotrypsinogen B expression strain before induction, while lane 2 shows the same strain after 4 hrs of IPTG induced expression. The chymotrypsinogen B enzyme is indicated by the arrow. The 3 other proteases are as indicated in the paired lanes. The figure indicates the actual expressions of the proteases have been obtained.

### EXAMPLE 1 (COMPARATIVE)

### Expression of Porcine Cationic Trypsin in Escherichia coli

The 231 amino acid porcine cationic trypsinogen sequence is obtainable from Swissprot file P00761 where the first 8 amino acids constitute the pro sequence that is cleaved of to produce the active enzyme trypsin as shown in FIG. 1.

The mature cationic trypsin protein sequence was translated to DNA sequence using Escherichia coli most frequently used anti codons using the codon usage table shown in FIG. 2.

The gene sequence was also controlled for the accuracy of the protein sequence and the presence of dyad symmetries that could interfere with transcription and the sequence was modified to remove the strongest structures. SphI and NsiI restriction sites were added to the 5′ and 3′ ends, respectively, to allow gene synthesis and cloning. Additionally a SapI restriction site was introduced at the 5′ end of the trypsin gene to allow cloning into plasmid pTwin2 (New England Biolabs). In this construction the cationic trypsinogen sequence is fused to the intein in pTwin2 and which after auto cleavage will release the cationic trypsinogen enzyme. The final sequence is given in FIG. 3.

This gene can be synthesised directly from overlapping oligonucleotides and then cloned into either of the cloning vectors pGEM5 or pGEM7 (Promega) and the DNA sequence may confirmed by DNA sequence analysis. The efficiency of cloning is improved using 3′ overhangs at the extremities due to oligonucleotide synthesis progressing from 3′ to 5′, hence ensuring that the 3′ end is complete (cloning using 5′ overhangs suffers as not all oligonucleotides reach the correct 5′ end). The final plasmid was then digested with the restriction enzymes SapI+NsiI and cloned into pTwin2 digested with SapI+PstI to give the plasmid shown in FIG. 4.

pTwin2 contains a mini-intein derived from the Synechocystis sp dnaB (Wu, H. et al., 1998. Biochim. Biophys. Acta. 1387:422-432) that has been engineered to undergo pH and temperature dependent cleavage at its C-terminus (Mathys, S., et al., 1999, Gene. 231:1-13). Inteins are peptide sequences sometimes found within proteins that are auto-catalytically removed to create the final active enzyme. This allows the purification of enzymes with any amino acid at the amino-terminus and not restricted to methionine.

SapI cleaves in this manner:

5′ . . . GCTCTTCN
3′ . . . CGAGAAGNNNN

The intein-trypsin fusion protein (FIG. 5) may be expressed from this plasmid or transferred into another expression plasmid such as pET24 or one of the numerous expression plasmids for E. coli. Expression may be achieved similar to the method described by Kiraly, O., et al., 2006, Protein Expr. Purif. 48:104-111. The plasmid pTwin2 uses the strong T7 promoter that is inducible by isopropyl 1-thio β D-galactopyranoside (IPTG) in an appropriate host strain such as ER2566 (New England Biolabs). Bacterial cells carrying the plasmid pTwin2-trypsin are cultivated in LB medium containing 100 μg/ml ampicillin for plasmid selection at 37° C. with aeration. At an optical density of approximately 0.5-0.7 OD₆₀₀, IPTG is added to a final concentration of 0.3-0.5 mM and the culture incubated at 15° C. for a further 16 h. Alternative conditions could be 37° C. for 2 h or 30° C. for 6 h depending on the toxicity of the expressed protein. After this time the cells are harvested by centrifugation (may be frozen at −20° C. until use).

The cells are suspended in 0.1 M Tris-HCl (pH 8.0), 5 mM K-EDTA and the cells are disrupted by sonication. The inclusion bodies containing the intein-trypsin fusion protein are then collected by centrifugation at 18,000 g for 5 minutes. The pellet was washed twice with the above buffer and then dissolved in the denaturing buffer containing 4 M guanidine-HCl, 0.1 M Tris-HCl (pH 8.0), 2 mM K-EDTA and 30 mM dithiothreitol at 37° C. for 30 minutes. Denatured proteins are then rapidly diluted 100× by adding refolding buffer (0.9 M guanidine-HCl, 0.1 M Tris-HCl (pH 8.0), 2 mM K-EDTA and 1 mM L-cysteine, 1 mM L-cystine) and are stirred under argon for 5 minutes and are incubated at 4° C. for 16 h. This solution was diluted in an equal volume of 0.4 M NaCl, centrifuged at 20,000 g for 15 minutes and the supernatant was loaded onto an ecotin affinity column. The column was washed with 20 mM Tris-HCl (pH 8.0), 0.2 M NaCl and the intein-trypsin fusion protein eluted with 50 mM Tris-HCl (pH 8.0). Cleavage of the intein from the trypsin can be achieved by incubating the fusion protein at 25° C. in 20 mM HEPES or Tris-HCl (pH 7.0), containing 500 mM NaCl, and 1 mM EDTA for 16 h. The mature trypsin may be further purified from the intein protein using the ecotin affinity column.

Alternatively, the intein cleavage may be done on the ecotin affinity column, washed and the purified subsequently trypsin eluted.

Alternatively, the gene expression could be performed in a strain of E. coli deficient in thioredoxin reductase to create a reducing environment to favour the formation of disulphide bonds and the direct production of an active enzyme without the need to denature and refold the enzyme (Verheyden, G., et al., 2000. J. Chromatogr. B Biomed. Sci. Appl. 737:213-224.).

Alternatively, a hexahistidine-tail could be engineered at the amino terminus to allow affinity purification using a Ni-NTA-agarose column.

Alternatively, the intein could be replaced by yeast ubiquitin, the recombinant protein purified and the ubiquitin removed using the purified yeast YUH1 enzyme.

### EXAMPLES 2-4

Anionic trypsinogen (comparative), Chymotrypsinogen B and Chymotrypsin C can be prepared in accordance with what is described above.

For anionic trypsinogen, reference is made to FIGS. 6-9.

For chymotrypsinogen B reference is made to FIGS. 10-12.

For chymotrypsinogen C reference is made to FIGS. 13-15.

### EXAMPLE 5

### Use of the Enzymes of Examples 1 to 4 to Partially Hydrolyse Whey Protein

254.6 kg of demineralised acidic whey powder, 91.3 kg whey protein concentrate obtained by ultrafiltration of sweet whey and 101.4 kg food-grade lactose are dispersed in 800 kg demineralised water at 60° C. The dispersion is placed in a double-walled reactor thermostatically controlled at 55° C. The dispersion has a dry matter content of 30.1% and a pH of 6.4. The pH is increased to 7.8 by addition of a 20% aqueous dispersion of Ca(OH)₂. 1 kg of a mixture of trypsin and chymotrypsin produced as described above (strength 6 AU/g, trypsin:chymotrypsin activity ratio 15:1-20:1 in USP) dispersed in a 0.01M aqueous solution of HCl is then added at 5 to 10° C. to initiate the hydrolysis. If zymogen forms of trypsin and/or chymotrypsin are used, these may be activated by the addition of proteinases, as it is well known to those of skill in the art. The initial rapid fall in pH is then stopped, the pH being maintained at 7.3 using a pH-stat by automatic compensation with a 2N aqueous KOH solution.

Hydrolysis is continued for 3 hours at 55° C./pH 7.3 after which the pH is increased to 7.6 by adjustment of the pH-stat to the new value. The hydrolysate is passed through a plate-type hear exchanger where it is rapidly heated to 90° C., then to a dwell tube (flow rate 7.5 l/minute, tube volume 401 g, residence time 5 minutes) and then into a second plate-type heat exchanger where it is cooled to 55° C. The coiled hydrolysate is pumped at a rate of 7.5 l/minute through a T valve into a dwell tube 0.025 m in diameter for a volume of 150 l which corresponds to a residence time of 20 minutes over the entire length of the tube. A further 1 kg of the mixture of trypsin and chymotrypsin is pumped into the hydrolysate stream through the T valve at the entrance to the dwell tube at a rate of 6 l/hour. After pre-heating to 80° C. with a dwell time of 5 minutes the hydrolysate (which has undergone a total dwell time of 20 minutes) is pumped into a UHT steriliser where it is heated to 125° C. over a period of 2 minutes. After cooling, the hydrolysate is spray dried. The powder thus obtained comprises, by weight, 23% peptides, 68% lactose, 4% ash, 2% fats and 3% moisture. The degree of hydrolysis calculated as nitrogen×100/total nitrogen (Nt) is 185 and Nt is 3.56%.

Analysis by SDS-PAGE confirms the absence of protein bands. In particular, no bands corresponding to bovine serum albumin, alpha-lactalbumin, beta-lactoglobulin or the H and L chains of IgG are observed.

### EXAMPLE 6

### Preparation of Infant Formula Using the Partial Whey Hydrolysate of Example 5

The procedure of Example 5 is followed up to completion of the second hydrolysis. The hydrolysate is passed to a thermostatically controlled tank and held at 60° C. during the addition of an equivalent quantity of a solution of maltodextrin and starch having a dry matter content of 50% with mineral salts dissolved in demineralised water. The mixture is heated to 75° C. in a plate-type heat exchanger. A mixture of palm olein, coconut oil, safflower oil, lecithin and fat soluble vitamins is melted at 65° C. and added to the hydrolysate mixture in a quantity corresponding to 10% of the hydrolysate mixture. The complete mixture is pre-heated to 80° C. for 5 minutes and then to 125° C. for 2 minutes by direct injection of steam. The heat-treated mixture is cooled to 70° C. in an expansion vessel, homogenised in two stages first at 20 MPa and then at 5 MPa and cooled to 10° C. first in a plate-type heat exchanger and then in an intermediate storage tank. Then, a 10% solution of citric acid in demineralised water, water-soluble vitamins, oligo-elements ands taurine are added. Finally, the mixture is heated to 75° C., homogenised in one pass at 65-170 bar and spray dried. The resulting powder comprises by weight 12.5% peptides, 26% fats, 56.2% carbohydrates, 23% minerals and 3% moisture with traces of vitamins and oligo-elements.

1. Recombinant porcine chymotrypsin.

2. Chymotrypsin in accordance with claim 1 obtained from a synthetic gene.

3. Chymotrypsin in accordance with claim 1, wherein a part of the chymotrypsin is replaced, deleted, or added, while the protein still has at least 80% of the activity of porcine chymotrypsin.

4. DNA comprising at least one gene selected from the group consisting of recombinant and synthetic gene coding for porcine chymotrypsin.

5. DNA in accordance with claim 4, in the form of an expression vector.

6. DNA in accordance with claim 4, wherein a part of the chymotrypsin gene is replaced, deleted, or added, while the expressed protein still has at least 80% of the activity of porcine chymotrypsin.

7. A cell containing the DNA of claim 4 capable of expressing the protein coded for by the DNA of claim 4.

8. Cell according to claim 7, wherein the cell is a micro-organism.

9. Cell according to claim 8, wherein the micro-organism is a food grade micro-organism.

10. A method for digesting protein containing foodstuff or fractions thereof comprising using recombinant porcine chymotrypsin.

11. Method in accordance with claim 9 comprising the step of at least partially digesting protein fractions obtained from milk to improve the bodies ability to absorb the protein fraction after ingestion.

12. Chymotrypsin in accordance with claim 2, wherein a part of the chymotrypsin is replaced, deleted, or added, while the protein still has at least 80% of the activity of porcine chymotrypsin.

13. Cell according to claim 8, wherein the micro-organism is selected from the group consisting of a bacterial cell, a yeast cell, a plant cell, a fungi cell, an insect cell and a mammalian cell.
